(19) United States
(12) Patent Application Publication
(10) Pub. No.: US 2012/0116274 A1
(43) Pub. Date: May 10, 2012

(54) REMOTE UNATTENDED LOW LEVEL LIGHT THERAPY ORTHOPEDIC DEVICE, WEARING MEANS AND METHOD OF USE

(76) Inventor: Louis Grasso IV, Celebration, FL (US)

(21) Appl. No.: 13/291,865

(22) Filed: Nov. 8, 2011

Related U.S. Application Data
(60) Provisional application No. 61/411,936, filed on Nov. 10, 2010.

(51) Int. Cl. A61N 5/06; A61F 5/01

(52) U.S. Cl. 602/2; 607/90

(57) ABSTRACT

A new and useful portable remote unattended low level light therapy (“LLLT”) orthopedic device that is lightweight, programmable, portable, affordable, able to be worn passively by the patient, able to operate unattended by a practitioner or physician, and can be used to administer multiple and/or extended treatment intervals outside the office setting. The device comprises a light therapy circuit with a power supply, controller circuit, LED driver circuit and an array of near infrared LEDs. Additionally the device comprises mode indicators and a program receiving port for programming by an external programming means. The entirety of the electrical components of the device are enclosed in a circuit housing and attached to a wearing means or brace. En toto, the device is used to direct low level light towards an indicated part of a patients anatomy in the field or orthopedic therapy.

### CROSS-REFERENCE TO RELATED APPLICATIONS

This application claims the benefit of U.S. Provisional Application No. 61/411,936, filing date Nov. 10, 2010.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable.

### TECHNICAL FIELD

The present invention is in the technical field of medical devices. More particularly, the present invention is in the technical field of orthopedic devices. More particularly, the present invention is in the technical field of orthopedic devices with integrated light therapy

### BACKGROUND OF THE INVENTION

Light therapy in the orthopedic setting entails administering doses of visible or near infrared light (“NIR”) to a target area on a patient's body. This therapeutic method is implemented to reduce pain, inflammation and edema, promote the healing of wounds, deep tissues and nerves, and prevent cell death and tissue damage. This treatment method has been known for over forty years since the invention of lasers and is presently conducted in the clinical or hospital setting with large, high-power devices which administer relatively high intensities of NIR for short durations of time. The current treatment and implementation methods arise from the historical need for large apparatuses which have larger dosage control modules and light emitting probes, and is predominantly based on laser technology.

In the applicant's experience, current forms of light therapy must be performed over multiple sessions, for lengths of time that are impractical in the office setting or for the patient to complete due to scheduling, cost and other concerns. There is a deficiency in the existing and prior art wherein there are no lightweight, programmable, remote portable light therapy devices that can be worn by the patient, utilized unattended by physician or outside of the medical office, or for protracted or repeated periods of time.

Although smaller light therapy devices exist, they are still very expensive, large, and heavy—having a unit console body approximately one-half meter cubed, with separate probe or wand and power supply. These light therapy devices must be placed on a fixed surface next to a patient who is typically laying in a supine or prone position. The device must be plugged in to a wall outlet or other high voltage source. A practitioner must then administer the therapy by controlling a wand or probe which is tethered to the console. These devices are less than ideal for the patient and practitioner to use. Also, because they are quite expensive, it makes it unlikely that a treatment office will have more than one device.

There is a portable rechargeable therapeutic device disclosed in U.S. Patent Application Publication US 2007/0073366 A1. The device disclosed in the application is smaller than current devices, intended for home use and is essentially a flashlight. However, the oblong shape of this device does not solve the problem wherein use requires a practitioner or operator throughout treatment—even if said operator is the patient herself. This is essentially a probe similar in shape to those on existing larger light therapy devices. It must be held in hand and requires active manipulation during use. Overall, this device does not seem to successfully address ease of use, the ability to use the device unattended or the ability to administer treatment while the patient passively wears the device.

In the applicant's experience, there is a need for a light therapy orthopedic device which is i) lightweight, ii) programmable, iii) portable, iv) affordable, v) able to be worn passively by the patient, vi) able to operate unattended by a practitioner or physician, and vii) may be used to administer multiple and/or extended treatment intervals outside of the office setting.

### SUMMARY OF THE INVENTION

The present invention provides a new and useful portable remote unattended low level light therapy (“LLLT”) orthopedic device that is lightweight, programmable, portable, affordable, able to be worn passively by the patient, able to operate unattended by a practitioner or physician, and can be used to administer multiple and/or extended treatment intervals outside the office setting. This device is believed to be useful to deliver low levels of visible or NIR light to a localized area of the patient's anatomy to reduce pain, inflammation and edema, promote the healing of wounds, deep tissues and nerves, and prevent cell death and tissue damage in the patient. Indicated and proven uses include treatment of inflammatory disorders such as tendonitis, bursitis, epicondylitis, as well as disorders that involve a decrease in local blood flow such as diabetic vascular insufficiency in the feet. Modern research also points to increases in pain relief, increased cellular activity, localized blood flow, soft tissue repair, and bone fracture healing.

More specifically this device is believed to be useful in cases where longer or repeated sessions of light therapy are indicated. This device is believed to allow the patient to continue receiving treatment at intervals and durations that would be prohibitive or difficult to accomplish in the current paradigm and office setting. The present invention achieves its result by combining two treatment methods: orthopedic bracing and LLLT. The device supplements current bracing techniques with use of NIR light emitting diode (“LED”) light. By combing the bio-mechanical support of an orthopedic brace with the therapeutic effects of LLLT, recovery time of a wide range of musculo-skeletal disorders is greatly improved.

In one of its basic embodiments, the present invention is an orthopedic device which comprises an array of LLLT LEDs, that when arranged in a pattern and mounted onto a medium, can be shaped and sized to precisely conform to anatomical structures which are to be irradiated and are the subject of therapy. Said medium can be a circuit board and may be flexible. An embodiment of this invention incorporates said light arrays into an orthopedic brace or a wearing means, as indicated by the corresponding pathology (i.e. a carpal tunnel wrist brace with a LLLT LED array contacting and irradiating the carpal tunnel). In this manner, a pre-programmed treatment regimen of LLLT can be administered by wearing the corresponding orthopedic brace, which itself bio-mechanically benefits and supports the user.

In another embodiment of the present invention, the device may be used in other bracing and bandaging applications; some wearing means are proprietary while others are already available in the market. The currently available means with which the device can be used include Ace-type bandages, adhesive strips, and existing braces (i.e. low-back/neck braces, ankle braces, wrist braces, tennis elbow braces, and the like).

When the device is used for treatment, a wearing means is selected as indicated by the 100 pathology and desired results—for example a wrist brace is elected for treatment of carpal tunnel syndrome. The device is then powered via the powering means or power supply. The controller circuit is programmed for duration and interval corresponding to the desired therapy. The device is then attached to a wearing means or brace. The physician or patient can then fit the wearing means to the anatomy of the patient. The device is then turned on to implement the therapy.

In another embodiment of the invention, use of the device can be augmented by the optional steps of physician follow up care and reprogramming of the control circuit.

In another embodiment of the invention, use of the device can also include replacement or recharging of the powering means.

After use and at the end of the indicated treatment cycle, the device is turned off, and removed from the patient.

Further features and objectives of the present invention will become apparent form the following detailed description and the accompanying drawings.

### BRIEF DESCRIPTION OF THE DRAWINGS

FIG. 1 is a block diagram of a light therapy circuit of a remote unattended low level light therapy device according to the present invention;

FIG. 2 is a perspective view of a remote unattended low level light therapy device according to the present invention;

FIG. 3 is a perspective view of an alternative embodiment of the device;

FIG. 4 is a perspective view of an alternative embodiment of the device;

FIG. 5 is a front view of the device;

FIG. 6 is a front view of an alternative embodiment of the device;

FIG. 7 is a front view of an alternative embodiment of the device;

FIG. 8 is a back view of the device;

FIG. 9 is a side elevation view of the device; and

FIG. 10 is a view of the device in the environment when in use.

### DETAILED DESCRIPTION OF THE INVENTION

As described above, the present invention provides a new and useful portable remote unattended low level light therapy orthopedic device which is lightweight, portable, programmable, affordable, able to be worn passively by the patient, able to operate unattended by a practitioner or physician, and can be used to administer multiple and/or extended treatment intervals outside the office setting. The following description and accompanying drawings disclose at least one version of such a device.

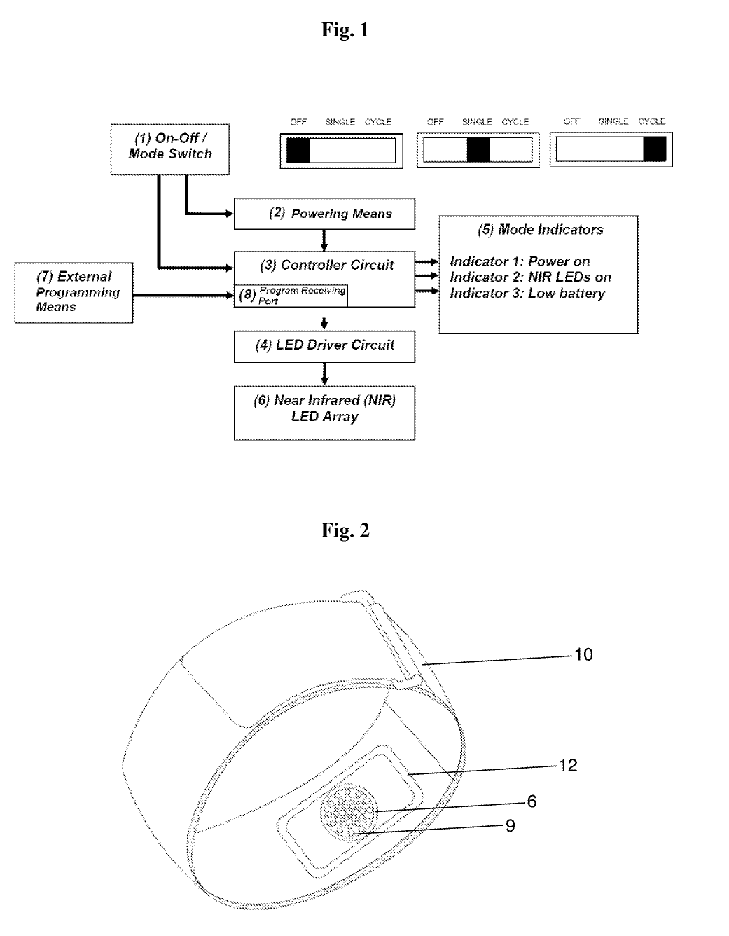

Referring to FIG. 1, an on-off/mode switch 1 serves two functions: (a) it applies power to the device through a powering means 2 which may be a battery, an AC wall adapter type power supply, or combination of the two, and, optionally, (b) the switch 1 determines the mode 140 of operation (single duration or repetitive cycling) of the device. Once power is applied, a microprocessor-based controller circuit 3 cycles a LED driver circuit 4 and a near infrared (NIR) LED array 6, for either a single predetermined ON period (single mode) or continuously cycles between predetermined ON and OFF periods (cycle mode). The array 6 consists of a plurality of LEDs 9 that operate in the 600 nm to 1000 nm range and a power density of about 1 mW/cm2 to 5000 mW/cm2. The LEDs 9 may be mounted on a medium, which can be a series of interconnected rigid or flexible circuit boards. The controller circuit 3 also monitors the battery status, if used as the power supply 2 and drives a plurality of mode indicators 5. The mode indicators 5 visually display device operating modes including: power on, NIR LEDS operating, and low battery. Optionally, an external programming means 7 can be attached to the device via a program receiving port 8 and used to set the device's on and off periods, and other parameters.

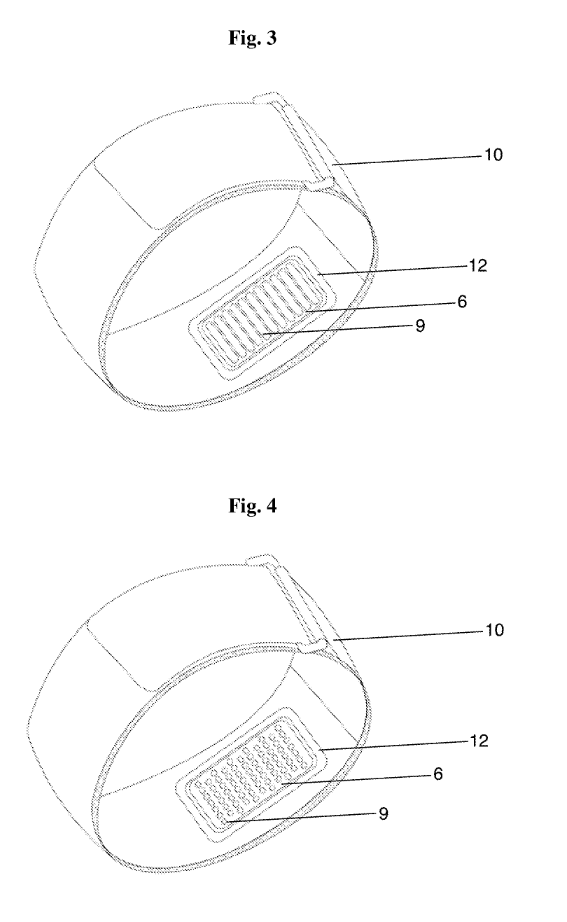

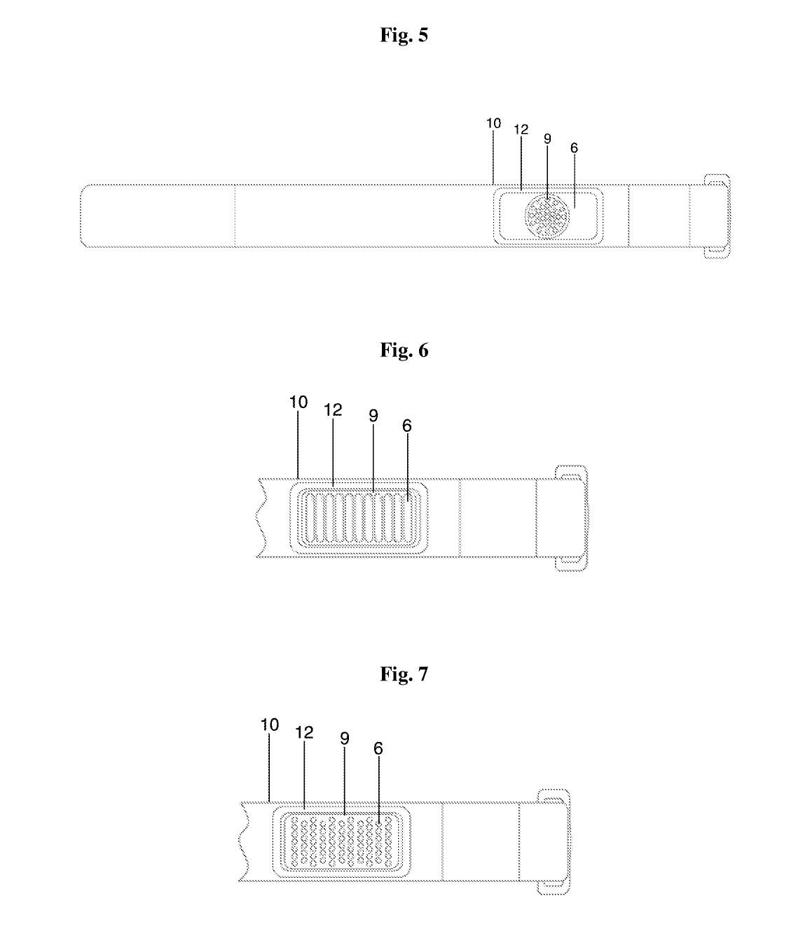

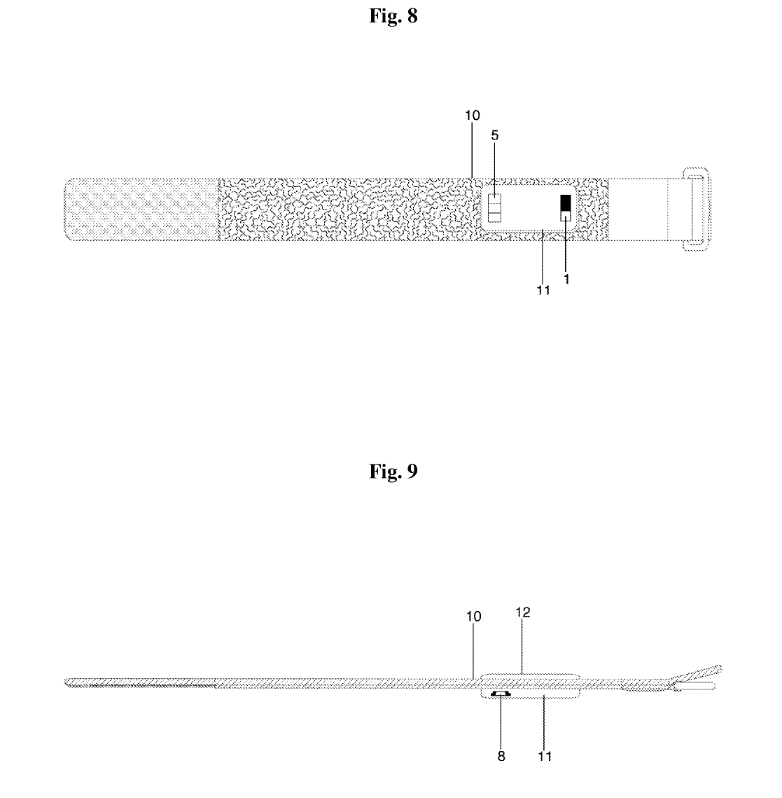

Referring now to the invention in more detail, in FIG. 2 to FIG. 10 there is shown a new and useful portable remote unattended low level light therapy orthopedic device comprising a NIR LED array 6 which emits light in the 600 nm to 1000 nm spectrum range; ideally focused in the 810 nm to 830 nm range for use with inflammatory pathologies, and 630 nm to 670 nm for promoting the healing of wounds. The array 6 is comprised of a plurality of individual LEDs 9. These LEDs 9 are electronically connected to a LED driver circuit 4 and arranged as to achieve optimal exposure levels of about 1 mW/cm2 to 100 mW/cm2. In one embodiment the LEDs 9 exhibit exposure levels of about 600 nm to 1000 nm, with a power density of about 1 mW/cm2 to 5000 mW/cm2. In one embodiment of the invention the LEDs 9 are evenly spaced.

The LEDs 9 are arranged into an LED array 6 and are mounted onto a medium that will vary in size and shape according to its anatomical application. The medium is a circuit board and can be flexible. The LEDs 9 in the array 6 are controlled by a controller circuit 3 and can be housed with the powering means 2. The entire assembly of the electronic components of the device are enclosed in the circuit housing 11 and attached to the wearing means 10 by a mounting means 12. The mounting means 12 can be a bracket, fasteners like hook and loop tape, or straps incorporated in the orthopedic brace or wearing means 10.

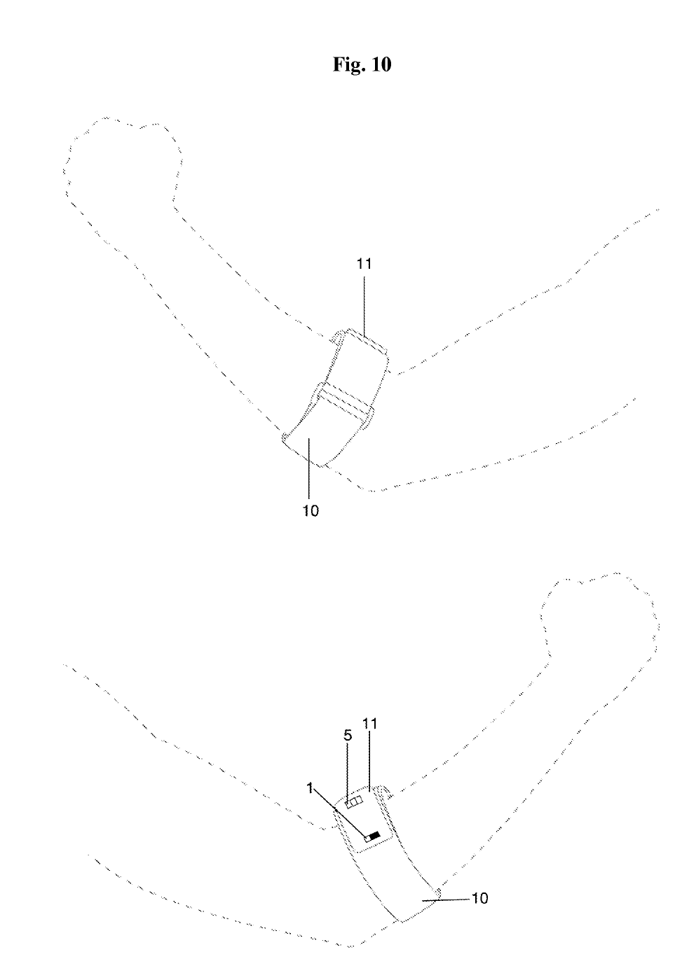

Referring now to the invention in more detail, in FIG. 9 to FIG. 10 the power supply 2 is low-profile and is enclosed in a circuit housing 11, as to prevent snagging, and can be mounted with a mounting means 12 anywhere on the wearing means 10 that does not exert pressure onto the braced region of the patient's anatomy to mitigate dermal or anatomical irritation.

In another embodiment, the mode indicators 5, on-off/mode switch 1, and program receiving port 8 are optionally mounted in the circuit housing 11 for visibility and ease of use. The mode indicators 5 are visually simple and will display several modes including: on—timed auto exposure, on—single exposure, and off. Optionally the program receiving port 8 may be a mini-USB port.

In another embodiment, the external programming means 7 can optionally be used to program the duration, intensity, and interval of each treatment as indicated. For example, a knee brace treatment may require one hour of LED exposure, while a carpal tunnel brace related treatment may need only ten minutes. Optionally, programming is accomplished by proprietary 180 software.

When the device is in use by the patient, it should fit comfortably and yet snugly enough to stay in place. It is easy to put on and take off, is pleasant to wear, and protects the patient from irritation.

Although very narrow claims are presented herein, it should be recognized the scope of this invention is much broader than presented by the claims. It is intended that broader claims will be submitted in an application that claims the benefit of priority from this application.

Insofar as the description above and the accompanying drawing disclose any additional subject matter that is not within the scope of the claims below, the inventions are not dedicated to the public and the right to file one or more applications to claim such additional inventions is reserved.

The previously described versions of the present invention have many advantages, including and without limitation, the properties of being i) lightweight, ii) portable, iii) programmable, iv) affordable, v) able to be worn passively by the patient, vi) able to operate unattended by a practitioner or physician, and vii) used to administer multiple and/or extended treatment intervals outside of the office setting. The device of the present invention is believed to accomplish all of the foregoing objectives. The invention does not require that all the advantageous features and all the advantages need to be incorporated into every embodiment of the invention.

Although the present invention has been described in considerable detail with reference to certain preferred versions thereof, other versions are possible. Therefore the spirit and scope of the appended claims should not be limited to the description of the preferred versions contained therein.

The reader's attention is directed to all papers and documents which are filed concurrently with this specification and which are open to public inspection with this specification, and the contents of all such papers and documents are incorporated herein by reference.

All the features disclosed in this specification may be replaced by alternative features serving the same, equivalent or similar purpose, unless expressly stated otherwise. Thus, unless expressly stated otherwise, each feature disclosed is one example only of a generic series of equivalent or similar features.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. As for “means for” elements, the applicant intends to encompass within the language any structure presently existing or developed in the future that performs the same function. The invention should therefore not be limited by the above described embodiment, method, and examples, but by all embodiments and methods within the scope and spirit of the invention.

What is claimed is:

1. A device for low light level therapy which comprises:
a) a powering means for delivering power to the device;
b) a controller circuit electrically connected to said powering means;
c) a LED driver circuit electrically connected to timing circuit;
d) a Near Infrared LED array, comprised of a plurality of LEDs electrically connected to led driver circuit;
e) a circuit housing surrounding the array and enclosing the electronic components of the device; and
f) a wearing means mounted to the circuit housing.

2. The device of claim 1, wherein the LEDs operate in a wavelength primarily of about 600 nm to about 1000 nm and a power density of about 1 mW/cm2 to about 100 mW/cm2.

3. The device of claim 1, wherein the LEDs operate in a wavelength primarily of about 600 nm to 1000 nm and a power density of about 1 mW/cm2 to 5000 mW/cm2.

4. The device of claim 1, wherein LEDs operate in a wavelength primarily of about 810 nm to about 830 nm and provides a power density of about 1 mW/cm2 to about 100 mW/cm2.

5. The device of claim 1, wherein LEDs operate in a wavelength primarily of about 810 nm to about 830 nm and a power density of about 1 mW/cm2 to 5000 mW/cm2.

6. The device of claim 1, wherein LEDs operate in a wavelength primarily of about 610 nm to about 700 nm and provides a power density of about 1 mW/cm2 to about 100 mW/cm2.

7. The device of claim 1, wherein LEDs operate in a wavelength primarily of about 610 nm to about 700 nm and provides a power density of about 1 mw/cm2 to 5000 mW/cm2.

8. The device of claim 1, wherein LEDS are mounted on a plurality of rigid circuit boards.

9. The device of claim 1, wherein LEDs are mounted on a flexible circuit board.

10. The device of claim 1, further comprising an on/off switch electrically connected to said powering means.

11. The device of claim 1, further comprising mode indicators, electrically connected to said controller circuit.

12. The device of claim 1, further comprising a mounting means to attach the circuit housing to the wearing means.

13. The device of claim 1, wherein controller circuit is microprocessor based.

14. The device of claim 1, further comprising a program receiving port electrically connected to timing circuit.

15. The device of claim 14, further comprising an external programming means removeably connected to program receiving port.

16. The device of claim 1, wherein said wearing means is an orthopedic brace.

17. The device of claim 1, wherein said wearing means is a bandage.

18. The device of claim 1, wherein said powering means is a battery.

19. A battery as in claim 18, wherein the battery is rechargeable.

20. A program receiving port as in claim 14, wherein the port is a USB input.

21. A method of using low light level therapy device which comprises:
a) selecting a wearing means as indicated by the pathology and desired results;
b) providing power to the device;
c) programming the controller circuit duration and interval as required by the desired therapy;
d) attaching the device to the wearing means;
e) fitting the wearing means onto the patient; and
f) turning the device on.

22. Method of use of claim 21, further comprising verifying the operation of device.

23. Method of use of claim 21, further comprising turning device off.

24. Method of use of claim 21, further comprising recharging the power source.

25. Method of use of claim 21, further comprising replacing the power source.

26. Method of use of claim 21, further comprising re-programming the controller circuit.

27. Method of use in claim 21, further comprising removing the wearing means.

28. Method of use in claim 21, further comprising removing the device from the wearing means.
